(19) United States
(12) Patent Application Publication
(10) Pub. No.: US 2024/0225758 A1
(43) Pub. Date: Jul. 11, 2024

(54) MULTI-ARM SURGICAL ROBOTIC PLATFORM

(71) Applicant: Mazor Robotics Ltd., Caesarea (IL)

(72) Inventors: Eli Zehavi, Tel-Aviv (IL); Moshe Shoham, Hoshaya (IL); Yonatan Ushpizin, Glil Yam (IL); Yizhaq Shmayahu, Ramat Hasharon (IL)

(21) Appl. No.: 18/558,750

(22) PCT Filed: May 22, 2022
(86) PCT No.: PCT/IL2022/050530

§ 371 Date: Nov. 3, 2023

Related U.S. Application Data
(60) Provisional application No. 63/193,430, filed on May 26, 2021.

(51) Int. Cl. A61B 34/30; A61B 34/00; A61B 90/00

(52) U.S. Cl. CPC A61B 34/30; A61B 34/70; A61B 90/361

(57) ABSTRACT

Multi-arm surgical robotic platforms and systems are provided. The platforms and systems may comprise a support structure or a frame and an operating table mounted to the support structure or the frame. A plurality of robotic arms may be mounted to the support structure or the frame and may be capable of manipulating an emitter and a detector to obtain between and including 0 to 360-degree imaging of a patient on the operating table.

### FIELD

The present technology generally relates to surgical robots, and relates more particularly to a multi-arm surgical robotic platform.

### BACKGROUND

Surgical robots may assist a surgeon or other medical provider in carrying out a surgical procedure, or may complete one or more surgical procedures autonomously. Imaging may be used by a medical provider for diagnostic and/or therapeutic purposes. The imaging may be provided by imaging devices operated by the surgical robots.

### SUMMARY

Example aspects of the present disclosure include:

A multi-arm surgical robotic platform according to at least one embodiment of the present disclosure comprises a support structure comprising an upper wall, a lower wall, and two sidewalls, the support structure defining a central aperture; an operating table mounted to the support structure and extending at least partially into the central aperture; a plurality of robotic arms, including a first robotic arm mounted to a first one of the two sidewalls, a second robotic arm mounted to a second one of the two sidewalls, and a third robotic arm mounted to the lower wall; wherein the plurality of robotic arms are capable of manipulating an emitter and a detector of an imaging device to provide between and including 0 to 360-degree imaging of a patient positioned on the operating table.

Any of the aspects herein, wherein each of the plurality of robotic arms is an accurate robotic arm.

Any of the aspects herein, wherein the operating table is mounted to the support structure such that a pose of the operating table relative to the support structure is selectively adjustable.

Any of the aspects herein, wherein the operating table is detachably mounted to the support structure.

Any of the aspects herein, further comprising a tool carousel mounted to a position on the upper wall, above the operating table.

Any of the aspects herein, further comprising an imaging device mounted to the upper wall.

Any of the aspects herein, wherein the imaging device corresponds to a navigation system.

Any of the aspects herein, wherein the imaging device is a depth camera.

Any of the aspects herein, further comprising: a controller comprising: a processor; and a memory storing instructions for execution by the processor that, when executed, cause the processor to: control the plurality of robotic arms to manipulate the emitter and the detector of the imaging device to capture between and including 0 to 360-degree imaging of the patient positioned on the operating table.

Any of the aspects herein, further comprising: a plurality of robotic arm sensors, each of the plurality of robotic arm sensors configured to detect at least one of force, torque, or position; and an operating table sensor.

Any of the aspects herein, further comprising a controller configured to receive information from the plurality of robotic arm sensors and the operating table sensor and to control each of the plurality of robotic arms in a single coordinate system.

Any of the aspects herein, wherein at least one of the plurality of robotic arms comprises an end effector configured to selectively grasp and release surgical tools.

Any of the aspects herein, wherein the support structure is fixedly securable to an operating room wall.

Any of the aspects herein, wherein at least one of the first robotic arm, the second robotic arm, or the third robotic arm is capable of selectively disengaging an imaging device and engaging a surgical tool.

Any of the aspects herein, wherein at least one of the first robotic arm, the second robotic arm, or the third robotic arm is capable of selectively disengaging a surgical tool and engaging an imaging device.

A surgical robotic platform according to at least one embodiment of the present disclosure comprises a support structure having an upper member, a lower member, and side members; an operating table mounted to the support structure and having a longitudinal axis, the support structure surrounding the longitudinal axis; a first robotic arm mounted to a first side member; a second robotic arm mounted to a second side member; a third robotic arm mounted to the lower member, underneath the operating table; and a controller configured to control each of the first, second, and third robotic arms in a single coordinate system.

Any of the aspects herein, wherein the controller is configured to control the third robotic arm and at least one of the first or second robotic arms to manipulate an emitter and a detector to obtain between and including 0 to 360-degree imaging of a patient on the operating table.

Any of the aspects herein, further comprising a navigation camera, wherein the controller is configured to generate navigation guidance based on information detected by the navigation camera.

Any of the aspects herein, further comprising a plurality of sensors configured to detect forces exerted on one or more of the operating table, the first robotic arm, the second robotic arm, the third robotic arm, and the support structure.

Any of the aspects herein, further comprising a tool carousel secured to the support structure, the tool carousel configured to hold a plurality of surgical tools.

Any of the aspects herein, wherein at least one of the first robotic arm, the second robotic arm, or the third robotic arm is capable of selectively depositing a first surgical tool in the tool carousel and engaging a second surgical tool from the tool carousel.

Any of the aspects herein, wherein at least one of the first robotic arm, the second robotic arm, or the third robotic arm is capable of selectively disengaging an imaging device and engaging a surgical tool.

Any of the aspects herein, wherein at least one of the first robotic arm, the second robotic arm, or the third robotic arm is capable of selectively disengaging a surgical tool and engaging an imaging device.

A surgical robotic system according to at least one embodiment of the present disclosure comprises a frame; an operating table adjustably mounted to the frame; a plurality of robotic arms mounted to the frame and capable of manipulating an emitter and a detector to obtain between and including 0 to 360-degree imaging of a patient on the operating table; a navigation camera fixedly mounted to the frame; and a controller configured to control the plurality of robotic arms based at least in part on input received from the navigation camera.

A surgical robotic system according to at least one embodiment of the present disclosure comprises a frame; an operating table adjustably mounted to the frame; a plurality of robotic arms mounted to the frame and capable of manipulating an emitter and a detector to obtain between and including 0 to 360-degree imaging of a patient on the operating table; and a controller configured to control the plurality of robotic arms based at least in part on input received from the navigation camera.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is an image of a robotic platform according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Surgical procedures frequently involve the use of various and/or multiple components such as imaging devices and surgical tools. In the case of X-ray based imaging a radiation source and a detector may be used and placed on opposite sides of the imaged organ. An extent of the imaging volume accessible and within reach of one or more surgical tools depends on a position and orientation of robotic arm mounts.

At least one embodiment disclosed includes a multi-arm robotic system calibrated to provide and use a shared coordinate system for all robotic arms. The system may include a setup utilizing multiple horizontally oriented robotic joints (e.g., wall mounts) with at least one robotic arm placed above a surgical table and at least one robotic arm placed below or underneath the table. At least one arm may be oriented horizontally using a wall mount. Each robotic arm can hold a surgical device or an imaging device at any particular time.

Integrating imaging and, for example, interventional radiology capabilities can be used to optimize imaging robotically, to support automated procedural steps and to widen a spectrum of procedures supported by robotic surgery. Such integration may shorten a procedure time and enhance procedural outcome.

Embodiments of the present disclosure provide technical solutions to one or more of the problems of (1) integration of multiple robotic arms, (2) providing between and including 0 to 360-degree patient imaging, (3) operating and orienting multiple robotic arms to perform one or more tasks simultaneously and/or sequentially, and (4) increase patient safety.

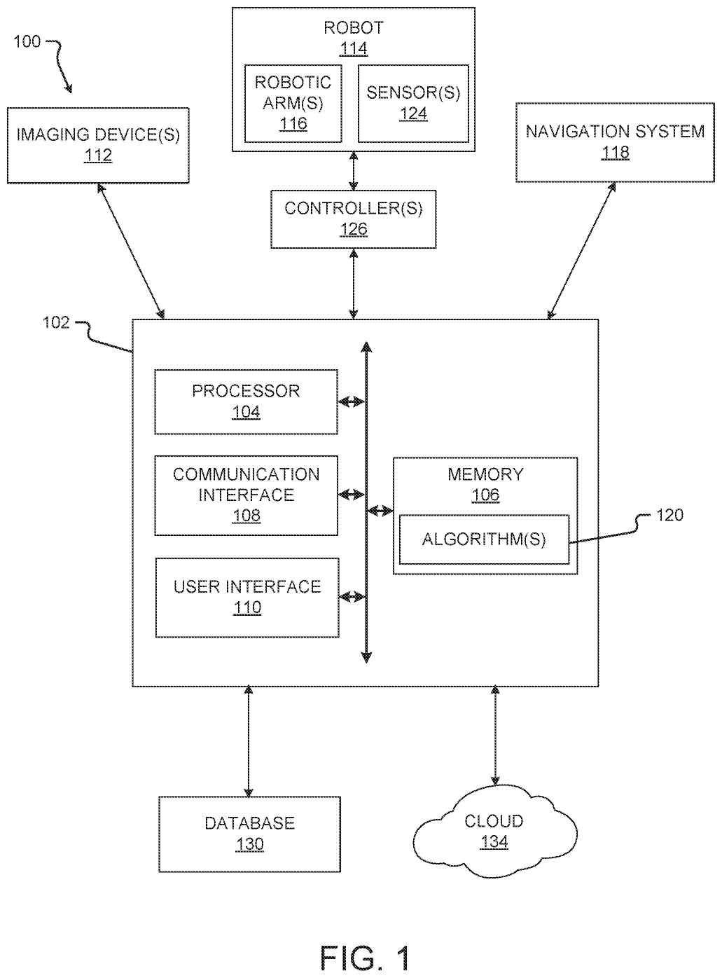

Turning first to FIG. 1, a block diagram of a system 100 according to at least one embodiment of the present disclosure is shown. The system 100 may be used to control one or more robotic arms and/or carry out one or more other aspects of one or more of the methods disclosed herein. The system 100 comprises a computing device 102, one or more imaging devices 112, a robot 114, a navigation system 118, one or more controllers 126, a database 130, and/or a cloud or other network 134. Systems according to other embodiments of the present disclosure may comprise more or fewer components than the system 100. For example, the system 100 may not include the imaging device 112, the robot 114, the navigation system 118, the controller 126, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the controller 126, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 300 described herein, or of any other methods. The memory 106 may store, for example, one or more algorithms 120. Such instructions or algorithms may, in some embodiments, be organized into one or more applications, modules, packages, layers, or engines. Alternatively or additionally, the memory 106 may store other types of data (e.g., machine learning modes, artificial neural networks, etc.) that can be processed by the processor 104 to carry out the various method and features described herein. Thus, although various components of memory 106 are described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, the controller 126, and/or the cloud 134.

The computing device 102 may also comprise a communication interface 108. The communication interface 108 may be used for receiving image data or other information from an external source (such as the imaging device 112, the robot 114, the navigation system 118, the controller 126, the database 130, the cloud 134, and/or any other system or component not part of the system 100), and/or for transmitting instructions, images, or other information to an external system or device (e.g., another computing device 102, the imaging device 112, the robot 114, the navigation system 118, the controller 126, the database 130, the cloud 134, and/or any other system or component not part of the system 100). The communication interface 108 may comprise one or more wired interfaces (e.g., a USB port, an ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer or emitter and receiver or detector, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), a depth camera (e.g., a camera that may use, for example, Time of Flight to judge width, height, area, volume, depth, and/or distance of an object) or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 (or any number of components that comprise the imaging device 112, for example, an emitter separate from a detector) at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure.

The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more robotic arm sensors 124. The sensor 124 may be a force sensor, configured to detect a force applied on a corresponding robotic arm 116 (e.g., whether via an end effector of the robotic arm 116, a tool held by an end effector of the robotic arm 116, or otherwise). The sensor 124 may be a position sensor, a proximity sensor, a magnetometer, or an accelerometer that enables the processor 104 (or a processor of the robot 114) or the controller 126 to determine a precise pose in space of the robotic arm (as well as any object or element held by or secured to the robotic arm. In some embodiments, the sensor 124 may be a linear encoder, a rotary encoder, an incremental encoder, or an Inertial Measurement Unit (IMU). In still other embodiments, the sensor 124 may be an imaging sensor. Other types of sensors may also be used as the sensor 124. The one or more sensors 124 may be positioned, for example, on the robotic arm 116 or elsewhere or may be integrated with the robotic arm 116.

Data from the sensor(s) 124 may be provided to a processor of the robot 114, to the processor 104 of the computing device 102, to the controller 126, and/or to the navigation system 118. The data may be used, for example, to calculate a position in space of the robotic arm 116 relative to one or more coordinate systems. In other examples, the data may be used to calculate a reactionary force of the robotic arm 116 based on, for example, a force received by the robotic arm 116. The calculation may be based not just on data received from the sensor(s) 124, but also on data or information (such as, for example, physical dimensions) about, for example, the robot 114 or a portion thereof, or any other relevant object, which data or information may be stored, for example, in a memory 106 of a computing device 102 or in any other memory.

The robotic arm(s) 116 may be accurate robotic arm(s). Such accurate robotic arm(s) 116 may be able to repeatedly return to a pose with a high degree of accuracy and/or may be caused to orient to a new pose with a high degree of accuracy. Further, a pose of the robotic arm 116, whether determined by a processor using sensor data obtained from the sensor 124 or otherwise, may be determined with a high degree of accuracy.

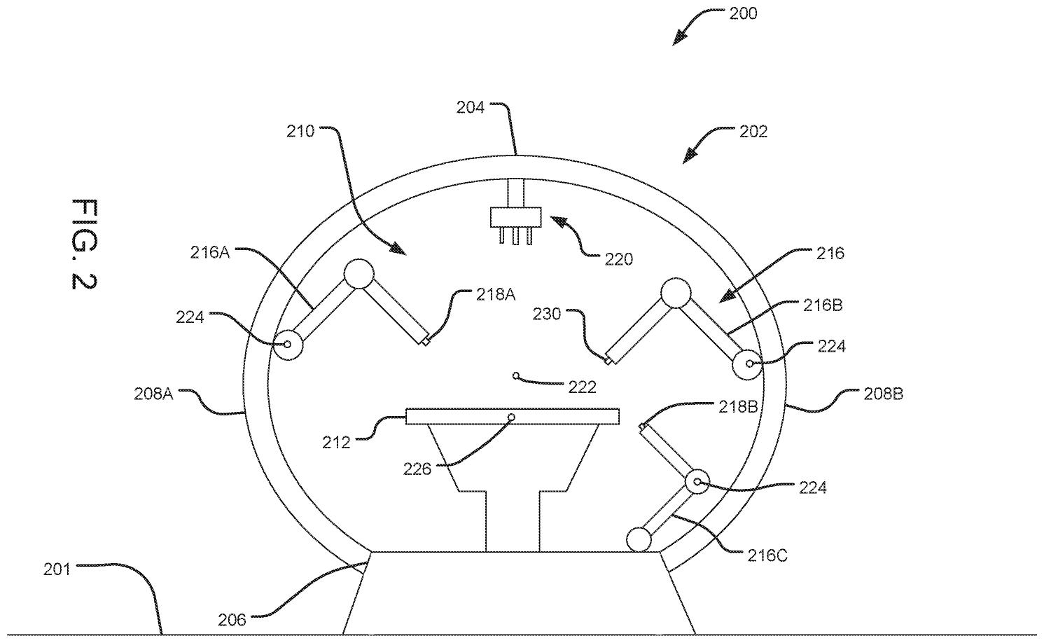

In some embodiments, reference markers (i.e., navigation markers) may be placed on the robot 114 (including, e.g., on the robotic arm 116), the imaging device 112, a support structure such as a support structure 202 (described with respect to FIG. 2), a table such as a table 212 (described with respect to FIG. 2) or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras RF. In some embodiments, the navigation system may comprise one or more electromagnetic sensors. In various embodiments, the navigation system 118 may be used to track a position and orientation (i.e., pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

In the illustrated embodiment, the system 100 includes the controller 126, though in some embodiments the system 100 may not include the controller 126. The controller 126 may be an electronic, a mechanical, or an electro-mechanical controller. The controller 126 may comprise or may be any processor described herein. The controller 126 may comprise a memory storing instructions for executing any of the functions or methods described herein as being carried out by the controller 126. In some embodiments, the controller 126 may be configured to simply convert signals received from the computing device 102 (e.g., via a communication interface 108) into commands for operating the robot 114 and/or the robot arm 116. In other embodiments, the controller 126 may be configured to process and/or convert signals received from, the sensor 124, the navigation system 118, and/or the robot 114. Further, the controller 126 may receive signals from one or more sources (e.g., the sensor 124, the navigation system 118, and/or the robot 114) and may output signals to one or more sources.

The controller 126 is configured to control a plurality of robotic arms 116 or a single robotic arm 116. In embodiments where the robotic arm 116 includes a plurality of robotic arms, one controller 126 may control the plurality of robotic arms 116 in some instances, and in other instances, one or more controllers 126 may control the plurality of robotic arms 116. In other embodiments, each robotic arm 116 may be controlled by a corresponding controller 126. The controller 126 is also configured to cause one or more robotic arms 116 to orient to a pose, to orient and/or operate a tool, to change a tool, to orient and/or operate an imaging device such as the imaging device 112, and/or to orient and/or operate any component or tool of the system 100 or the operating room.

The system 100 or similar systems may be used, for example, to carry out one or more aspects of any of the method 300 described herein. The system 100 may also be used in connection with a robotic platform such as a robotic platform 200, described below. The system 100 or similar systems may also be used for other purposes.

FIG. 2 shows a robotic platform 200 according to at least one embodiment of the present disclosure. The platform 200 includes a support structure 202 and an operating table 212. The structure 202 comprises an upper wall or member 204, a lower wall or member 206, and a pair of sidewalls or members 208A, 208B. The operating table 212 includes a longitudinal axis 222 and the structure 202 surrounds the longitudinal axis 222. In other words, the upper wall 204, the lower wall 206, and the pair of sidewalls 208A, 208B each surround the longitudinal axis 222 and the table 212. In some embodiments, the structure 202 surrounds the longitudinal axis 222 entirely. The upper wall 204, the lower wall 206, and the pair of sidewalls 208A, 208B may also define an aperture 210 that the table 212 extends at least partially into. In some embodiments the table 212 is mounted to the support structure 202. In other embodiments, the table 212 may not be mounted to the support structure 202 and may be mounted elsewhere.

In some embodiments, the structure 202 is fixedly securable to an operating room wall 201 (such as, for example, a ground surface of an operating room). In other embodiments, the structure 202 may be releasably securable to the operating room wall 201 or may be a standalone component that is simply supported by the operating room wall 201. In some embodiments, the table 212 may be mounted to the structure 202. In other embodiments, the table 212 may be releasably mounted to the structure 202. In still other embodiments, the table 212 may not be attached to the structure 202. In such embodiments, the table 212 may be supported and/or mounted to an operating room wall, for example. In embodiments where the table 212 is mounted to the structure 202 (whether detachably mounted or permanently mounted), the table 212 may be mounted to the structure 202 such that a pose of the table 212 relative to the structure 202 is selectively adjustable.

The table 212 may be any operating table 212 configured to support a patient during a surgical procedure. The table 212 may include any accessories mounted to or otherwise coupled to the table 212 such as, for example, a bed rail, a bed rail adaptor, an arm rest, an extender, or the like. The operating table 212 may be stationary or may be operable to maneuver a patient (e.g., the operating table 212 may be able to move). In some embodiments, the table 212 has two positioning degrees of freedom and one rotational degree of freedom, which allows positioning of the specific anatomy of the patient anywhere in space (within a volume defined by the limits of movement of the table 212). For example, the table 212 can slide forward and backward and from side to side, and can tilt (e.g., around an axis positioned between the head and foot of the table 212, and extending from one side of the table 212 to the other) and/or roll (e.g., around an axis positioned between the two sides of the table 212, and extending from the head of the table 212 to the foot thereof). In other embodiments, the table 212 can bend at one or more areas (which bending may be possible due to, for example, the use of a flexible surface for the table 212, or by physically separating one portion of the table 212 from another portion of the table 212 and moving the two portions independently). In at least some embodiments, the table 212 may be manually moved or manipulated by, for example, a surgeon or other user, or the table 212 may comprise one or more motors, actuators, and/or other mechanisms configured to enable movement and/or manipulation of the table 212 by a processor such as the processor 104.

The platform 200 also comprises a plurality of robotic arms 216. Each of the robotic arms 216 may be the same as or similar to the robotic arms 116. The plurality of robotic arms 216 may be dimensioned so that one or more robotic arm may reach various portions of a patient such as, for example, a patient spine. As previously described, each of the robotic arms 216 may be an accurate robotic arm 216. Such accurate robotic arm(s) 216 may be able to repeatedly return to a pose with a high degree of accuracy and/or may be caused to orient to a new pose with a high degree of accuracy. Further, a pose of the robotic arm 216, whether determined by a processor (such as the processor 104) using sensor data obtained from a robotic arm sensor 224 (which may be the same as or similar to the sensor 124) or otherwise, may be determined with a high degree of accuracy.

In some embodiments, the plurality of robotic arms 216 may be independent of and unattached to the table 212. In other words, the plurality of robotic arms 216 may be manipulated and moved separately from the table 212. In such embodiments, the plurality of robotic arms 216 may be secured to one or more of a floor, an wall, and/or ceiling of an operating room, or to any structure of the platform 200. In other embodiments, the plurality of robotic arms 216 may be attached directly to the table 212. In some embodiments, the plurality of robotic arms 216 may be attached to the table 212 with a gantry. In other embodiments, the plurality of robotic arms 216 may be attached to the table 212 without a gantry.

In the illustrated embodiment, a first robotic arm 216A is attached to or otherwise mounted to the first sidewall 208A and a second robotic arm 216B is attached to or otherwise mounted to the second sidewall 208B. It will be appreciated that the platform 200 may have any number of robotic arms including one robotic arm, two robotic arms, or more than two robotic arms. In the illustrated embodiment, the first robotic arm 216A and the second robotic arm 216B are also shown positioned on opposite sides of the aperture 210. In other embodiments, the first robotic arm 216A and the second robotic arm 216B may be positioned anywhere along the aperture 210. In some embodiments, a third robotic arm 216C may be mounted to the lower wall 206. In still further embodiments, any number of robotic arms 216 may be positioned anywhere on any component of the platform 200 or surgical room (e.g., on the table 212, the support structure 202, and/or an operating room wall).

The plurality of robotic arms 216 may be capable of or operable to manipulate an imaging device such as the imaging device 112. It will be appreciated that the imaging device 112 may also be independent of the robotic arms 216. For example, the imaging device 112 may be mounted to the upper wall 204 or to any component (e.g., the table 212, the support structure 202, the plurality of robotic arms 216, etc.) of the platform 200 or operating room. In some embodiments, the imaging device may comprise an emitter 218A and a detector 218B. In such embodiments, one of the robotic arms 216, for example, the first robotic arm 216A, the second robotic arm 216B, or the third robotic arm 216C, may orient and operate the emitter 218A and another one of the robotic arms 216, for example, the first robotic arm 216A, the second robotic arm 216B, or the third robotic arm 216C, may orient and operate the detector 218B. In some instances, the plurality of robotic arms 216 may orient the emitter 218A and the detector 218B so as to provide between and including 0 to 360-degree imaging of a patient positioned on the table 212. In some embodiments, the emitter 218A and the detector 218B may provide between and including 0 to 360-degree imaging of a patient and a surgical tool during, for example, a surgical task or procedure.

In some embodiments, one or more robotic arms 216 of the plurality of robotic arms 216 may comprise an end effector 230 configured to selectively grasp and release surgical tools. For example, a robotic arm such as the second robotic arm 216B may be configured to selectively grasp and release surgical tools. In some embodiments, the tools may be held in a tool carousel 220, described in more detail below. In other embodiments, the tools may be positioned on a table, a platform, or other surface for supporting the tools. In still other embodiments, the tools may be received by the end effector 230 from a user such as a surgeon or other medical provider.

The platform 200 may also comprise the tool carousel 220. In some embodiments, the platform 200 may not include the tool carousel 220. In the illustrated embodiment, the tool carousel 220 is mounted to the upper wall 204 and above the table 212. In other embodiments, the tool carousel 220 may be mounted to any component (e.g., the table 212, the support structure 202, the plurality of robotic arms 216, etc.) of the platform 200 or any component of an operating room. The carousel 220 in some instances may be rotatable. In some embodiments a motor may rotate the carousel 220. In other embodiments, the carousel 220 may be rotated manually by, for example, a user such as a surgeon or other medical provider. In some embodiments, instructions may be generated by, for example, a processor such as the processor 104 and transmitted to the motor to cause the motor to automatically rotate the carousel 220. In some instances, the instructions may be based on a surgical plan. In other embodiments, the motor may rotate based on input received from the user.

The carousel 220 may be configured to hold or otherwise support one or more tools or instructions. It will be appreciated that the carousel 220 can hold one tool or instrument, two tools and/or instruments, or more than two tools and/or instruments. The carousel 220 may also be configured to hold and/or lock the tool such that the tool will not fall out of or otherwise move out of the carousel 220. The carousel 220 may also lock the tool such that the tool cannot be retrieved unless instructions (which may be generated by the processor 104) to unlock the tool are transmitted to the carousel 220, a robotic arm 216 unlocks the tool, or a user unlocks the tool.

The platform 200 may also comprise a navigation system such as the navigation system 118. The navigation system 118 may include an imaging device such as the imaging device 112 mounted to the upper wall 204. In other embodiments, the imaging device of the navigation system 118 may be mounted to any component (e.g., the table 212, the support structure 202, the plurality of robotic arms 216, etc.) of the platform 200.

The platform 200 may also comprise a controller such as the controller 126 to control the plurality of robotic arms 216. The controller 126 may cause the plurality of robotic arms 216 to perform one or more tasks simultaneously and/or sequentially. For example, the controller 126 may simultaneously cause a first robotic arm to operate a tool to perform a surgical procedure and a second robotic arm to operate an imaging device to obtain images of the tool. The controller 126 may control the plurality of robotic arms 216 in one or a single coordinate system. By controlling the plurality of robotic arms 216 in one coordinate system, the controller 126 may, for example, avoid collisions between the robotic arms 216 as the controller knows where each robotic arm 216 is located in the coordinate space. The controller 126 may also control the plurality of robotic arms 216 to perform any tasks or operate any component of the system 100 and/or the platform 200 or any component of any system and/or platform. For example, the controller 126 may cause the robotic arms 216 to orient and operate the imaging device 112 to provide between and including 0 to 360-imaging of a patient. In another example, the controller 126 may cause the robotic arms 216 to orient and/or operate any tool or imaging device. In yet another example, the controller 126 may cause one or more robotic arms 216 to depositing a tool in the carousel 220 and engaging another tool in the carousel 220, thereby switching tools. In still another example, the controller 126 may cause one or more robotic arms 216 to selectively disengage an imaging device and engage a surgical tool (whether from the carousel 220 or elsewhere). Similarly, in other examples, the controller 126 may cause one or more robotic arms 216 to selectively disengage a surgical tool and engage an imaging device.

The platform 200 may also comprise a plurality of robotic arm sensors 224. The sensors 224 may correspond to transducers that are configured to convert physical phenomena into an electrical signal that is capable of being processed by the controller 126. Non-limiting examples of sensors 224 include gyroscopic sensors, accelerometers, strain gauges, impact sensors, vibration detectors, etc. The sensor 224 may be the same as or similar to sensors 124. Each sensor 224 may be configured to detect at least one of a force, a torque, or a position of a corresponding robotic arm 216 and yield corresponding sensor data. The sensor data may be used to provide feedback to the controller 126 or a processor such as the processor 104 of a computing device such as the computing device 102. The controller 126 or the processor 104 may use the feedback to control the plurality of robotic arms 216. For example, sensor data may indicate that a robotic arm 216 is experiencing an undesired force that may move the robotic arm 216 out of a desired position. The controller 126 may cause the robotic arm 216 to exert a reciprocal force and cause the robotic arm 216 to move back into the desired position. In another example, sensor data may indicate that the robotic arm 216 is experiencing high forces and may result in the controller causing the robotic arm 216 to cease operations or generating a notification to the user. In yet another example, if an actual position of a robotic arm 216 obtained from the information does not match a predetermined position of the robotic arm 216, the controller 126 may cause the robotic arm 216 to orient or otherwise move to the predetermined position.

The platform 200 may also comprise an operating table sensor 226. The sensor 226 may be the same as or similar to the sensor 124. The sensor 226 may be configured to detect at least one of a force, a torque, or a position of the table 212 and yield corresponding sensor data. The sensor data may be used to provide feedback to the controller 126 or a processor such as the processor 104 of a computing device such as the computing device 102. The controller 126 or the processor 104 may use the feedback to control the plurality of robotic arms 216. For example, sensor data may indicate that the table 212 is experiencing undesired forces from a robotic arm 216. The controller 126 or the processor 104 may cause the robotic arm 216 to cease operations or reduce an amount of force exerted by the robotic arm 216 experienced by the table 212. In cases where the table 212 includes a motor, the controller 126 or the processor 104 may use the feedback to control the motor.

It will be appreciated that the controller 126 may receive sensor data from both the plurality of robotic sensors 224 and the operating table sensor 226. The controller 126 may control each of the plurality of robotic arms 216 and/or the operating table 212 based on the sensor data received. The controller 126 may also, in some embodiments, generate instructions for a user such as a surgeon or other medical provider based on the sensor data.

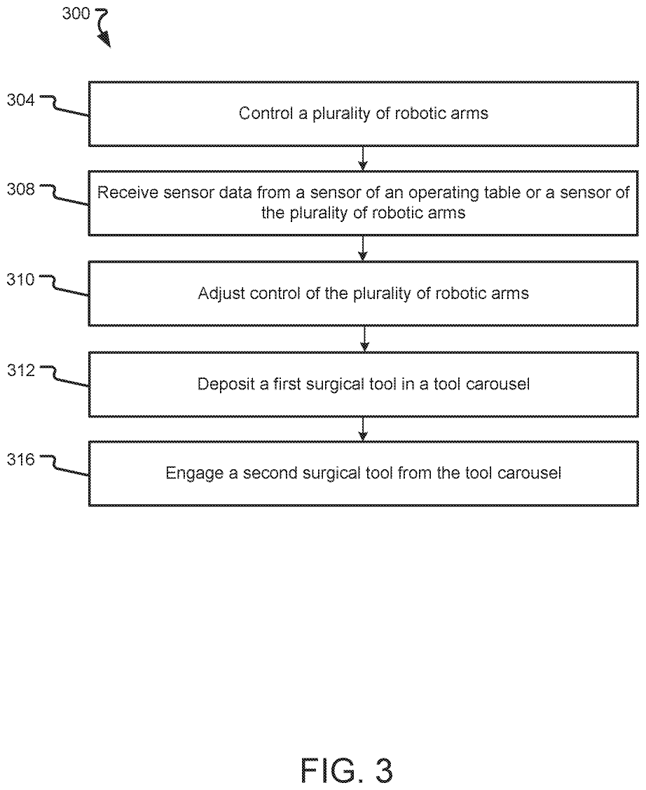

FIG. 3 depicts a method 300 that may be used, for example, for operating a surgical robotic platform. The surgical robotic platform may be the same as or similar to the surgical robotic platform 200. In some embodiments, the platform may be operated using a system such as the system 100 or otherwise used in conjunction with the system 100.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing instructions stored in a memory such as the memory 106. The instructions may correspond to one or more steps of the method 300 described below. The instructions may cause the processor to execute one or more algorithms, such as algorithms 120.

The method 300 comprises controlling a plurality of robotic arms (step 304). The plurality of robotic arms may be the same as or similar to the plurality of robotic arms 116, 216. The robotic arms may be components of a surgical robotic platform such as the platform 200. The platform may include a support structure such as the support structure 202 to which the robotic arms are mounted to. The support structure, in some embodiments, may form an aperture such as the aperture 210 and an operating table such as the operating table 212 may be disposed at least partially into the aperture.

In some embodiments the plurality of robotic arms may be controlled by a controller such as the controller 126. In other embodiments, the plurality of robotic arms may be controlled by, for example, a processor such as the processor 104 of a computing device such as the computing device 102. The plurality of robotic arms may be controlled in a single coordinate space. The single coordinate space may include a patient coordinate space, a robotic coordinate space, and a navigated coordinate space each correlated to a single space (whether the patient coordinate space, the robotic coordinate space, or the navigated coordinate space).

The step 304 may also comprise causing the plurality of robotic arms to manipulate an imaging device such as the imaging device 112. In some instances, the imaging device may include an emitter such as the emitter 218A and a detector such as the detector 218B. A first robotic arm such as the first robotic arm 216A may orient and control the emitter 218A and a second or third robotic arm such as the third robotic arm 216C may orient and control the detector 218b. In such embodiments, causing the plurality of robotic arms to manipulate the imaging device may include causing the first robotic arm to orient and control the emitter and the second robotic arm to orient and control the detector to provide between and including 0 to 360-degree imaging of a patient positioned on the operating table.

The step 304 may comprise causing the plurality of robotic arms to manipulate the imaging device to perform a scanning procedure. In some embodiments, the imaging device may include a source and a detector. The source may be held by a first robotic arm and the detector may be held by a second robotic arm. During the scanning procedure, the first robotic arm and the second robotic arm may change a relative orientation and position between the source and the detector to enhance image data obtained from the source and detector (e.g., causing the imaging device to zoom into a critical area, adjusting a resolution of image data obtained from the image device such as applying a multiresolution analysis or super resolution to the image data, or applying other algorithms to the image data).

The method 300 also comprises receiving sensor data from an operating table sensor of an operating table or one or more robotic arm sensors of the plurality of robotic arms (step 308). The operating table sensor may be the same as or similar to the operating table sensor 226. The robotic arm sensors may be the same as or similar to the robotic arm sensors 124, 224. Each of the operating table sensor or the robotic arm sensors (whether the table sensor and/or the robotic arm sensors) may be configured to detect at least one of a force, a torque, or a position of the table or a corresponding robotic arm, respectively.

The method also comprises adjusting the controlling of the plurality of robotic arms based on sensor data (step 310). The sensor data may be the forces detected in step 308, which may be used as feedback for controlling the plurality of robotic arms. For example, the data may be used to calculate a reactionary force of the robotic arm 114 based on, for example, a force received by the robotic arm. In the same examples, the controller may cause a corresponding robotic arm to exert the reactionary force in step 304.

The method 300 also comprises depositing a first surgical tool in a tool carousel (step 312). The tool carousel may be the same as or similar to the tool carousel 220. The first surgical tool may be any tool or instrument such as, for example, a screw, a screwdriver, a tulip or receiver, a rod, a knife, an ablation tool, etc. The first surgical tool may be deposited by an end effector of a robotic arm of the plurality of robotic arms. The end effector may be the same as or similar to the end effector 230. The robotic arm may be, for example, the first robotic arm 216A, the second robotic arm 216B, or the third robotic arm 216C. In such embodiments, instructions may be automatically generated by, for example, a processor such as the processor 104 and transmitted to the robotic arm. The instructions may be based on, for example, a surgical plan. In other embodiments, a user such as a medical provider or surgeon may instruct the robotic arm to deposit the first surgical tool in the tool carousel.

The method 300 also comprises engaging a second surgical tool from the tool carousel (step 316). The second surgical tool may be engaged by the end effector of the robotic arm. In such embodiments, instructions may be automatically generated by, for example, the processor and transmitted to the robotic arm. The instructions may be based on, for example, a surgical plan. In other embodiments, a user such as a medical provider or surgeon may instruct the robotic arm to deposit the first surgical tool in the tool carousel.

It will be appreciated that steps 312 and 316 may be repeated any number of times for any number of tools. For example, the second tool may be deposited in the tool carousel, a third tool may be engaged, and so on and so forth. It will also be appreciated that steps 312 and/or 316 may be executed by a robotic arm of a plurality of robotic arms, while one or more other robotic arms perform other tasks. For example, the robotic arm may deposit the first surgical tool and/or engage the second surgical tool while a pair of robotic arms orient and operate a detector and an emitter. In such embodiments, the plurality of robotic arms may operate in a single coordinate system.

The present disclosure encompasses embodiments of the method 300 that comprise more or fewer steps than those described above, and/or one or more steps that are different than the steps described above.

As noted above, the present disclosure encompasses methods with fewer than all of the steps identified in FIG. 3 (and the corresponding description of the method 300), as well as methods that include additional steps beyond those identified in FIG. 3 (and the corresponding description of the method 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A multi-arm surgical robotic platform comprising:
a support structure comprising an upper wall, a lower wall, and two sidewalls, the support structure defining a central aperture;
an operating table mounted to the support structure and extending at least partially into the central aperture;
a plurality of robotic arms, including a first robotic arm mounted to a first one of the two sidewalls, a second robotic arm mounted to a second one of the two sidewalls, and a third robotic arm mounted to the lower wall;
wherein the plurality of robotic arms are capable of manipulating an emitter and a detector of an imaging device to provide between and including 0 to 360-degree imaging of a patient positioned on the operating table.

2. The multi-arm surgical robotic platform of claim 1, wherein each of the plurality of robotic arms is an accurate robotic arm.

3. The multi-arm surgical robotic platform of claim 1, wherein the operating table is mounted to the support structure such that a pose of the operating table relative to the support structure is selectively adjustable.

4. The multi-arm surgical robotic platform of claim 1, wherein the operating table is detachably mounted to the support structure.

5. The multi-arm surgical robotic platform of claim 1, further comprising a tool carousel mounted to a position on the upper wall, above the operating table.

6. The multi-arm surgical robotic platform of claim 1, further comprising an imaging device mounted to the upper wall.

7. The multi-arm surgical robotic platform of claim 6, wherein the imaging device corresponds to a navigation system.

8. The multi-arm robotic platform of claim 6, wherein the imaging device is a depth camera.

9. The multi-arm robotic platform of claim 1, further comprising:
a controller comprising:
a processor; and
a memory storing instructions for execution by the processor that, when executed, cause the processor to:
control the plurality of robotic arms to manipulate the emitter and the detector of the imaging device to capture between and including 0 to 360-degree imaging of the patient positioned on the operating table.

10. The multi-arm robotic surgical platform of claim 1, further comprising:
a plurality of robotic arm sensors, each of the plurality of robotic arm sensors configured to detect at least one of force, torque, or position; and
an operating table sensor.

11. The multi-arm robotic surgical platform of claim 10, further comprising a controller configured to receive information from the plurality of robotic arm sensors and the operating table sensor and to control each of the plurality of robotic arms in a single coordinate system.

12. The multi-arm robotic surgical platform of claim 1, wherein at least one of the plurality of robotic arms comprises an end effector configured to selectively grasp and release surgical tools.

13. The multi-arm robotic surgical platform of claim 1, wherein the support structure is fixedly securable to an operating room wall.

14. The multi-arm robotic surgical platform of claim 1, wherein at least one of the first robotic arm, the second robotic arm, or the third robotic arm is capable of selectively disengaging an imaging device and engaging a surgical tool.

15. The surgical robotic platform of claim 14, wherein at least one of the first robotic arm, the second robotic arm, or the third robotic arm is capable of selectively disengaging a surgical tool and engaging an imaging device.

16. A surgical robotic platform comprising:
a support structure having an upper member, a lower member, and side members;
an operating table mounted to the support structure and having a longitudinal axis, the support structure surrounding the longitudinal axis;
a first robotic arm mounted to a first side member;
a second robotic arm mounted to a second side member;
a third robotic arm mounted to the lower member, underneath the operating table; and
a controller configured to control each of the first, second, and third robotic arms in a single coordinate system.

17. The surgical robotic platform of claim 16, wherein the controller is configured to control the third robotic arm and at least one of the first or second robotic arms to manipulate an emitter and a detector to obtain between and including 0 to 360-degree imaging of a patient on the operating table.

18. The surgical robotic platform of claim 16, further comprising a navigation camera, wherein the controller is configured to generate navigation guidance based on information detected by the navigation camera.

19. The surgical robotic platform of claim 16, further comprising a plurality of sensors configured to detect forces exerted on one or more of the operating table, the first robotic arm, the second robotic arm, the third robotic arm, and the support structure.

20. The surgical robotic platform of claim 16, further comprising a tool carousel secured to the support structure, the tool carousel configured to hold a plurality of surgical tools.

21. The surgical robotic platform of claim 20, wherein at least one of the first robotic arm, the second robotic arm, or the third robotic arm is capable of selectively depositing a first surgical tool in the tool carousel and engaging a second surgical tool from the tool carousel.

22. The surgical robotic platform of claim 16, wherein at least one of the first robotic arm, the second robotic arm, or the third robotic arm is capable of selectively disengaging an imaging device and engaging a surgical tool.

23. The surgical robotic platform of claim 16, wherein at least one of the first robotic arm, the second robotic arm, or the third robotic arm is capable of selectively disengaging a surgical tool and engaging an imaging device.

24. A surgical robotic system, comprising:
a frame;
an operating table adjustably mounted to the frame;
a plurality of robotic arms mounted to the frame and capable of manipulating an emitter and a detector to obtain between and including 0 to 360-degree imaging of a patient on the operating table;
a navigation camera fixedly mounted to the frame; and
a controller configured to control the plurality of robotic arms based at least in part on input received from the navigation camera.

25. A surgical robotic system, comprising:
a frame;
an operating table adjustably mounted to the frame;
a plurality of robotic arms mounted to the frame and capable of manipulating an emitter and a detector to obtain between and including 0 to 360-degree imaging of a patient on the operating table; and
a controller configured to control the plurality of robotic arms based at least in part on input received from the navigation camera.
